(19) United States
(12) Patent Application Publication
(10) Pub. No.: US 2023/0165826 A1
(43) Pub. Date: Jun. 1, 2023

(54) INHALATIONAL THERAPY FOR COVID-19

(71) Applicant: MEMORIAL SLOAN KETTERING CANCER CENTER, New York, NY (US)

(72) Inventors: Takeshi Irie, New York, NY (US); Ouathek Ouerfelli, Fort Lee, NJ (US)

(21) Appl. No.: 17/912,498

(22) PCT Filed: Mar. 18, 2021
(86) PCT No.: PCT/US2021/022997

§ 371 Date: Sep. 17, 2022

Related U.S. Application Data
(60) Provisional application No. 62/991,561, filed on Mar. 18, 2020.

(51) Int. Cl. A61K 31/245; A61K 9/00; A61K 47/40; A61P 31/14

(52) U.S. Cl. CPC A61K 31/245; A61K 9/0078; A61K 47/40; A61P 31/14

(57) ABSTRACT

The present invention is directed to compositions for administration of camostat mesylate or nafamostat mesylate to subjects by inhalational delivery, and to methods of treatment of COVID19 by administering such compositions to subjects by inhalational delivery.

### CROSS-REFERENCE TO RELATED APPLICATIONS

This application claims the benefit of priority of U.S. Provisional Pat. Application No. 62/991,561, filed on Mar. 18, 2020, the content of which is hereby incorporated by reference in its entirety.

### STATEMENT REGARDING FEDERALLY SPONSORED RESEARCH

This invention was made with government support under CA008748 and CA243895 awarded by the National Institutes of Health. The government has certain rights in the invention.

### INCORPORATION BY REFERENCE

For the purposes of only those jurisdictions that permit incorporation by reference, all of the references cited in this disclosure are hereby incorporated by reference in their entireties. In addition, any manufacturers’ instructions or catalogues for any products cited or mentioned herein are incorporated by reference. Documents incorporated by reference into this text, or any teachings therein, can be used in the practice of the present invention.

### BACKGROUND

Coronavirus disease 2019 (“COVID-19”) — which is caused by the SARS-CoV-2 coronavirus — was declared to have reached pandemic status by the World Health Organization (WHO) in March 2020. Mortality from COVID-19 is currently estimated to be around 2% in the US, although the mortality rate depends on numerous factors. It is expected that SARS-CoV-2 infection will cause significant loss of life globally, with current estimates exceeding 500 thousand deaths in the US alone, and may also incur significant morbidity and chronic illness in survivors of COVID-19. Although the rapid development, approval, and deployment of vaccines has great promise to curb the COVID-19 epidemic, those who become infected are still at risk of adverse outcomes and as such, there is an urgent need for therapeutic options for the treatment of COVID-19.

### SUMMARY OF THE INVENTION

Some of the main aspects of the present invention are summarized below. Additional aspects are described in the Detailed Description of the Invention, Examples, and Claims sections of this disclosure. The description in each section of this patent disclosure, regardless of any heading or sub-heading titles, is intended to be read in conjunction with all other sections. Furthermore, the various embodiments described in each section of this disclosure can be combined in various different ways, and all such combinations are intended to fall within the scope of the present invention.

Camostat mesylate is a protease inhibitor approved for clinical use in Japan for the treatment of chronic pancreatitis and postoperative esophagitis. Camostat mesylate was recently shown to be an inhibitor of a cellular protease (TMPRSS2) required for entry of the SARS-CoV-2 virus into lung cells in a study by Hoffmann et al. entitled “SARS-CoV-2 Cell Entry Depends on ACE2 and TMPRSS2 and Is Blocked by a Clinically Proven Protease Inhibitor. ” See Hoffmann et al., Cell, (2020), Vol. 181, pp. 1-10 (published online Mar. 5, 2020). Hoffman et al. found that camostat mesylate had only modest potency for blocking SARS-CoV-2 virus entry into lung cells - having an apparent EC₉₀ of 5-10 mM. This led us to hypothesize: (a) that systemic administration of camostat mesylate may cause significant off-target side effects at the doses required to block SARS-CoV-2 entry, and (b) that localized delivery of camostat (e.g., camostat mesylate) directly to affected lung tissue may provide therapeutic efficacy with reduced side-effects (inhalational delivery directly to the lung typically requires about 400 times less drug than is required for systemic delivery).

We also hypothesized that direct administration of camostat mesylate to the lungs in combination with systemic (e.g. oral or IV) delivery of camostat mesylate might be even more effective - with possible synergistic effects resulting from targeting both the lung and upper GI tract pathologies associated with COVID-19 disease.

Nafamostat mesylate is an analog of camostat mesylate, suggested to have similar activity against SARS-CoV-2 that has also been approved for use in human subjects. Therefore, we hypothesized that inhibition of SARS-CoV-2 viral entry via inhalational administration of nafamostat (e.g., nafamostat mesylate or nafamostat bismesylate) may also be clinically beneficial for treating COVID-19 disease. In June 2020, a Japanese collaborative group announced investigations of inhalational nafamostat mesylate for COVID-19 treatment (www.biospectrumasia.com/news/91/16087/j apan-explores-nafamostat-inhalation-formulation-for-COVID-19-treatment.html).

Gabexate mesylate is another camostat analog that may be active against SARS-CoV-2 entry to cells. The relative in vitro potency against SARS-CoV-2 reported for these three agents is nafamostat > camostat > gabexate.

Accordingly, we sought to develop various formulations of these agents suitable for inhalational administration to human subjects and suitable for use in treating COVID-19 by inhalational delivery. Compared to more conventional routes of administration (such as oral and parenteral routes) formulating drugs for inhalational delivery poses additional challenges, given the need for careful control of particle size and other key parameters. However, we were able to successfully formulate camostat and nafamostat - providing formulations that are stable at 4° C. and at ambient room temperature and have particle sizes and other properties that make them good candidates for inhalational administration. Building on this work, which is described in more detail in the Examples section of this patent disclosure, the present invention provides a variety of compositions suitable for inhalational delivery and methods of using such compositions in the treatment of COVID19.

Thus, in some embodiments the present invention provides a variety of compositions suitable for inhalational administration to subjects, wherein such compositions comprise: (a) camostat mesylate or nafamostat mesylate and (b) a β cyclodextrin (e.g., sulfobutyl-β-cyclodextrin or hydroxypropyl-β-cyclodextrin).

In other embodiments the present invention provides a variety of methods of treating COVID-19 in a subject, such methods comprising administering to a subject in need thereof an effective amount of a pharmaceutical composition comprising camostat mesylate or nafamostat mesylate.

Additional details of these and other embodiments of the present invention are provided and described in the Detailed Description, Examples and Claims sections of this patent application, which follow. Furthermore, it should be understood that variations and combinations of each of the embodiments described herein are contemplated and are intended to fall within the scope of the present invention.

### DETAILED DESCRIPTION

The sub-headings provided below, and throughout this patent disclosure, are not intended to denote limitations of the various aspects or embodiments of the invention, which are to be understood by reference to the specification as-a-whole. For example, this Detailed Description is intended to read in conjunction with, and to expand upon, the description provided in the Summary of the Invention section of this application.

### I. Definitions & Abbreviations

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Systeme International de Unites (SI) accepted form.

Numeric ranges provided herein are inclusive of the numbers defining the range. Where a numeric term is preceded by “about,” the term includes the stated number and values ±10% of the stated number.

Wherever embodiments are described with the language “comprising,” otherwise analogous embodiments described in terms of “consisting of′ and/or “consisting essentially of” are included.

As used herein the abbreviation “ACE2” refers to angiotensin converting enzymze 2.

As used herein the abbreviation “SBBCD” refers to sulfobutyl-β-cyclodextrin (also referred to in the art as sulfobutyl ether-β-cyclodextrin) which is also sold under the tradename Captisol® / CAPTISOL.

As used herein the abbreviation “HPBCD” refers to hydroxypropyl-β-cyclodextrin.

As used herein the abbreviation “WFI” refers to water for injection.

The terms “composition” and “formulation” are used interchangeably herein.

Various other terms are defined elsewhere in this patent disclosure, where used. Furthermore, terms that are not specifically defined herein may be more fully understood in the context in which the terms are used and/or by reference to the specification in its entirety. Where no explicit definition is provided all technical and scientific terms used herein have the meanings commonly understood by those of ordinary skill in the art to which this invention pertains.

### II. Active Agents

### A. Camostat

Several of the embodiments of the present invention involve the active agent camostat mesylate (i.e., 4-[[4-[(Aminoiminomethyl)amino]benzoyl]oxy]benzeneacetic acid 2-(dimethylamino)-2-oxoethyl ester methanesulfonate). Camostat mesylate is commercially available from multiple commercial sources - including SigmaAldrich (catalog # SML0057) and R&D Systems.

### B. Nafamostat

Several of the embodiments of the present invention involve the active agent nafamostat mesylate (i.e., 4-[(Aminoiminomethyl)amino]benzoic acid 6-(aminoiminomethyl)-2-naphthalenyl ester dimethanesulfonate), which is also referred to in the art as nafamostat bismesylate (the terms nafamostat mesylate and nafamostat bismesylate may be used interchangeably herein). Nafamostat mesylate is commercially available from multiple commercial sources - including from Sigma Aldrich (catalog # N0289).

### C. Gabexate

Several of the embodiments of the present invention involve the active agent gabexate mesylate (i.e., 4-[[6-[(Aminoiminomethyl)amino]-1-oxohexyl]oxyl]benzoic acid ethyl ester mesylate salt). Gabexate mesylate is commercially available from multiple commercial sources - including from Sigma Aldrich (catalog # G2417).

### III. Compositions

The present invention provides compositions comprising one or more of the active agents described above or elsewhere herein.

In some embodiments the compositions comprise one or more solubilizing agents, such as a cyclodextrin or water (e.g., water for injection or “WFI”).

In some embodiments, the present invention provides pharmaceutical compositions suitable for administration to human subjects by inhalational delivery, such compositions comprising: (a) camostat mesylate, nafamostat mesylate (e.g., nafamostat bismesylate), or gabexate mesylate and (b) a cyclodextrin.

In some embodiments, the present invention provides pharmaceutical compositions suitable for administration to human subjects by inhalational delivery, such compositions comprising: (a) camostat mesylate and (b) a cyclodextrin.

In some embodiments, the present invention provides pharmaceutical compositions suitable for administration to human subjects by inhalational delivery, such compositions comprising: nafamostat mesylate (e.g., nafamostat bis-mesylate) and (b) a cyclodextrin.

In some embodiments the cyclodextrin (CD) selected from the group consisting of: HPBCD, SBBCD, α-CD, β-CD, γ-CD, 2-hydroxypropyl-γ-CD (HPγCD), hydroxypropyl-β-cyclodextrin (HP-β-CD), sulfobutylether-β-cyclodextrin (SBE-β-CD, heptakis-2,3,6-tris-O-methyl β-CD (TRIMEB), heptakis-2,6-di-O-methyl-β-CD (DIMEB), randomly methylated beta-cyclodextrin, crystalline methylated beta-cyclodextrin, octasodium 6A,6B,6C,6D,6E,6F,6G,6H- -octakis-S-(2-carboxyethyl)-6A,6B,6C,6D,6E,6F,6G,6-octathio-γ-CD, and Epichlorohydrin-β-cyclodextrin.

In some embodiments the cyclodextrin is a β-cyclodextrin. In some such embodiments the β-cyclodextrin is SBBCD or HPBCD.

In some embodiments such compositions are aqueous solutions, e.g., solutions comprising water (e.g., water for injection (“WFI”)).

In some embodiments the compositions comprise a physiological saline.

In some embodiments the compositions are formulated to have concentrations such that are iso-osmotic with blood and extracellular fluids.

In some embodiments the compositions are aqueous solutions, comprising camostat mesylate or nafamostat mesylate at a concentration of about 30-60 mg/ml. In some such embodiments such compositions are aqueous solutions comprising camostat mesylate or nafamostat mesylate at a concentration of about 40-50 mg/ml. In some such embodiments such compositions are aqueous solutions comprising camostat mesylate or nafamostat mesylate at a concentration of about 45 mg/ml.

In some embodiments the compositions are aqueous solutions comprising about 5-15% w/v HPBCD. In some such embodiment such compositions are aqueous solutions comprising about 10% w/v HPBCD.

In some embodiments the compositions are aqueous solutions comprising about 5-15% w/v SBBCD. In some such embodiment such compositions are aqueous solutions comprising about 10% w/v HPBCD. about 12.5% w/v SBBCD.

In some embodiments the compositions further comprise one or more excipients suitable for inhalational delivery. Examples of such excipients include, but are not limited to, co-solvents, carriers, preservatives, chelating agents, buffers, pH regulators, tonicity regulators, amino -acids, salts, carbohydrates, polymers, and surfactants.

In some embodiments, including, in particular those in which delivery using an inhaler is desired, the compositions further comprise a propellant. Examples of such propellants include, but are not limited to, chlorofluorocarbons (CFCs) or hydrofluoroalkane (HFA), which are used for successful aerosolization and inhalational delivery of asthma medications.

In some embodiments the compositions are in aerosol form. In some such embodiments such aerosol forms comprise liquid droplets of from about 1 to about 10 microns in diameter. In some such embodiments such aerosol forms comprise liquid droplets of from about 1 to about 8 microns in diameter. In some such embodiments such aerosol forms comprise liquid droplets of from about 1 to about 6 microns in diameter. In some such embodiments such aerosol forms comprise liquid droplets of from about 1 to about 4 microns in diameter.

In some embodiments the compositions are in lyophilized form (as described in Example 3, these compositions can be successfully lyophilized - which increases their stability and shelf-life).

In some embodiments the compositions are stable at 4° C. In some such embodiments such compositions are stable at ambient room temperature (typically around 21° C.).

In some embodiments the compositions are in dry powder form.

In some embodiments the compositions are in atomisable form.

In some such embodiments the compositions are in particulate form.

In some embodiments the compositions are in micro-ionized form.

Examples of suitable inhalable forms of camostat mesylate include those described in U.S. Pat. Application No. 2012/0208882, the content of which is hereby incorporated by reference in its entirety.

In some embodiments the compositions comprise ethanol, which has been previously shown to improve the size of aerosolized particles to improve pulmonary drug delivery.

In some embodiments the compositions comprise a bulking agent, such as lactose, glucose or mannitol.

In some embodiments the compositions comprise liposomes.

In some embodiments the compositions comprise a polymer, such as polyethylene glycol (PEG), poly-lactic acid (PLA), poly-glycolic acid (PGA), a combination of poly-lactic/poly-glycolic acid (PLGA), or polyvinylpyrrolidone (also known as povidone or PVP).

In some embodiments the compositions comprise one or more “additional agents” - i.e., in addition to the “active agents” described above, that are useful to treat a SARS-CoV-2 infection or COVID19 or any symptom associated therewith. Examples of such “additional agents” include, but are not limited to, or remdesivir, an antibody or antibody-like molecule targeting the SARS-CoV-2 interaction with ACE2 (e.g., bamlanivimab or etesevimab), arformoterol, buphenine, clenbuterol, dopexamine, epinephrine, fenoterol, formoterol, isoetarine, isoprenaline, levosalbutamol, levalbuterol, orciprenaline, metaproterenol, pirbuterol, procaterol, ritodrine, salbutamol, albuterol, salmeterol, terbutaline, arbutamine, befunolol, bromoacetylalprenololmenthane, broxaterol, cimaterol, cirazoline, etilefrine, hexoprenaline, higenamine, isoxsuprine, mabuterol, methoxyphenamine, oxyfedrine, ractopamine, reproterol, rimiterol, tretoquinol, tulobuterol, zilpaterol, or zinterol, benzocaine, chloroprocaine, cocaine, cyclomethycaine, dimethocaine (larocaine), piperocaine, propoxycaine, procaine (novocaine), proparacaine, tetracaine (amethocaine), amide groups, lidocaine, articaine, bupivacaine, cinchocaine (dibucaine), etidocaine, levobupivacaine, mepivacaine, prilocaine, ropivacaine, trimecaine, tetrodotoxin, saxitoxin, neosaxitoxin, menthol, eugenol, spilanthol, aclidinium (tudorza pressair), glycopyrronium (seebri neohaler), ipratropium (atrovent), tiotropium (spiriva), and umeclidinium (incruse ellipta).

### IV. Methods of Treatment

The present invention provides various methods of treating COVID-19 in subjects in need thereof.

For example, in some embodiments the present invention provides a method of treating COVID-19, the method comprising administering to a subject in need thereof an effective amount of an inhalable pharmaceutical composition comprising camostat mesylate.

Similarly, in other embodiments the present invention provides a method of treating COVID-19, the method comprising administering to a subject in need thereof an effective amount of an inhalable pharmaceutical composition comprising nafamostat mesylate.

And in other embodiments the present invention provides a method of treating COVID-19, the method comprising administering to a subject in need thereof an effective amount of an inhalable pharmaceutical composition comprising gabexate mesylate.

And in yet other embodiments the present invention provides a method of treating COVID-19, the method comprising administering to a subject in need thereof an effective amount of any of the inhalable pharmaceutical compositions described above or elsewhere herein, such as those that comprise: (a) camostat mesylate, nafamostat mesylate, or gabexate mesylate and (b) a cyclodextrin.

As used herein, the terms “treat,” “treating,” and “treatment” refer achieving, and/or administering a composition to a subject to achieve, to a detectable degree, an improvement in one or more clinically relevant parameters associated with COVID-19 disease or SARS-CoV-2 infection in that subject. For example, the terms “treat,” “treating,” and “treatment” include, but are not limited to, inhibiting the activity of the cellular protease TMPRSS2 in lung cells, inhibiting entry of the SARS-CoV-2 virus into lung cells, inhibiting or reducing the severity of at least one symptom of COVID-19, slowing the development of one or more symptoms of COVID-19, reducing the duration of one or more symptoms of COVID-19, and the like. As used herein the terms “treat,” “treating,” and “treatment” encompass both preventive/prophylactic treatments and therapeutic treatments. In the case of prophylactic treatments, the methods and compositions provided herein can be used preventatively in subjects that do not yet exhibit any clear or detectable clinical indicators or symptoms of COVID-19 but that are believed to be at risk of developing such symptoms, for example due to infection with SARS-Cov-2 or contact with an individual infected with SARS-Cov-2. In the case of therapeutic treatments, the methods and compositions provided herein can be used in subjects that already exhibit one or more clinical symptoms of COVID-19. Typical clinical symptoms of COVID-19 are known to medical practitioners in the field and others skilled in the art, and include, for example, fever, cough, sore throat, shortness of breath, pneumonia, fatigue, body aches, muscle aches, loss of taste or smell, nausea, vomiting and diarrhea.

In some embodiments the methods of treatment provided by the present invention further comprise administration of an effective amount one or more “additional agents” (i.e., in addition to the “active agents” and compositions containing those agents, described herein) to a subject in need thereof. Such additional agents are described above under the “Compositions” sub-heading. Such additional agents can be administered by inhalational delivery, where appropriate, or by any other suitable route (e.g., intravenously, orally, etc.).

In some embodiments the methods of treatment provided by the present invention herein further comprise performing one or more additional medical interventions known to be useful for COVID-19 therapy and/or treatment of SAS-CoC-2 infection, including, but not limited to, methods useful for respiratory support - such as supply of oxygen, provision of mechanical ventilation, administration of steroids, etc. Similarly, in certain embodiments the methods of treatment provided herein may be employed together with procedures used to monitor disease status/progression.

In some embodiments the treatment methods described herein may be employed in conjunction with performing a diagnostic test to determine if the subject has COVID-19. For example, in some embodiments, prior to commencing treatment, a diagnostic assay is performed to determine if the subject has COVID-19.

### V. Subjects

As used herein the term “subject” encompasses all mammalian species, including, but not limited to, humans, non-human primates, dogs, cats, rodents (such as rats, mice and guinea pigs), cows, pigs, sheep, goats, horses, and the like - including all mammalian animal species used in animal husbandry, as well as animals kept as pets and in zoos, etc.

In preferred embodiments the subjects are human.

In some embodiments the subject has tested positive for SARS-CoV-2.

In some embodiments the subject is exhibiting one or more symptoms of COVID-19.

In some embodiments the subject is not exhibiting symptoms of COVID-19 but is believed to be as risk of developing COVID-19 symptoms, for example as a result of contact with an individual with a SARS-CoV-2 infection and/or COVID-19.

In some embodiments, the subject requires or is receiving respiratory support (e.g., supplemental oxygen and/or mechanical ventilation). In some embodiments the subject is intubated and/or on a ventilator.

In some embodiments the subject is critically ill.

In some embodiments the subject is elderly, has heart disease, has hypertension, has lung disease, has diabetes, has cancer, has liver dysfunction, has coagulation dysfunction or has organ failure, or is immunosuppressed or immunocompromised.

### VI. Administration & Dosages

In carrying out the treatment methods described herein, any suitable mode of inhalational administration can be used to deliver the active agents and compositions described herein.

In some embodiments the active agents and compositions described herein are administered to subjects using an inhalational device such as a nebulizer (e.g., jet nebulizer, vibrating mesh nebulizer, aerosol nebulizer, or compressor nebulizer), inhaler (e.g., metered dose inhaler, dry powder inhaler, or soft mist inhaler), atomizer, vaporizer (e.g., vaporizer pen), and the like. Such methods of administration can be performed inside or outside of a hospital setting (e.g., they can be self-administered by a subject, e.g., at home).

In some embodiments the active agents and compositions described herein are administered into a breathing circuit - such as that of a mechanical ventilator system or intubation system. In some such embodiments the composition is administered to the subject using a nebulizer or vaporizer in line with the inspiratory limb of a breathing circuit attached to an intubated patient supported on mechanical ventilatory support, or in line with and between the endotracheal tube and self-inflating bag of an intubated patient being manually ventilated by a health care provider. Such administration methods will typically be performed by a trained medical professional, e.g., in a hospital setting.

CO₂ absorbent agents or systems are often used to remove CO₂ from recirculated gas mixtures during ventilation/intubation procedures. Some of these absorbent agents and systems contain strong bases (such as sodium or potassium hydroxide), which can have deleterious effects on certain classes of drugs, such as ester drugs. Thus, in some embodiments the active agents and compositions described herein are administered into a breathing circuit (such as that of a mechanical ventilator system or intubation system) in the absence of a CO₂ absorbent agent or system. In such embodiments alternative methods for minimizing re-breathing of exhaled CO₂ can be employed, for example be using high flow / flow rates of gases.

As used herein the term “effective amount” refers to an amount of the specified “active agent” or composition or that is sufficient to achieve, or contribute towards achieving, an improvement in one or more of the clinically relevant parameters associated with COVID-19 disease or SARS-CoV-2 infection that are described in the “treatment” description above.

An appropriate “effective amount” in any individual case may be determined using standard techniques known in the art, such as dose escalation studies and studies performed to determine the EC₅₀ and/or maximum tolerated dose of an active agent. For example, in some embodiments an “effective amount” of an active agent may be calculated based on studies performed in vitro or, preferably, in vivo (e.g., preclinical animal studies or human clinical trials) to assess the efficacy of the active agent. Furthermore, the “effective amount” may be determined taking into account such factors as the desired route of administration (e.g. inhalational), the desired delivery device (e.g. a nebulizer), the desired frequency of dosing, the desired duration of dosing, and patient characteristics including age, body weight and the presence of any medical conditions affecting drug metabolism. Furthermore, an “effective amount” may be determined in the context of any co-administration method to be used. One of skill in the art can readily perform such dose-finding studies (whether using single agents or combinations of agents) to determine the appropriate “effective amount” in a given situation.

In some embodiments one or more of the active agents is used at approximately its maximum tolerated dose, for example as determined in a phase I clinical trials and/or in a dose escalation study. In some embodiments one or more of the active agents is used at about 90% of its maximum tolerated dose. In some embodiments one or more of the active agents is used at about 80% of its maximum tolerated dose. In some embodiments one or more of the active agents is used at about 70% of its maximum tolerated dose. In some embodiments one or more of the active agents is used at about 60% of its maximum tolerated dose. In some embodiments one or more of the active agents is used at about 50% of its maximum tolerated dose. In some embodiments one or more of the active agents is used at about 40% of its maximum tolerated dose. In some embodiments one or more of the active agents is used at about 30% of its maximum tolerated dose. In some embodiments one or more of the active agents is used at about 20% of its maximum tolerated dose.

The bioactivity of inhalational camostat has been demonstrated in guinea pig trachea to have an ED₅₀ of 3 mcg/kg (Coote et al., JPET, 2009 PMID 1919023). Coote et al., reported that inhalation of camostat could achieve bioactivity (in that case, enhancement of epithelial sodium channel activity, which impacts muco-ciliary clearance) that lasted for 5 hours after inhaled dosing.

In some embodiments the active agents or compositions of the invention may be delivered to subjects continuously during a course of treatment.

In some embodiments the active agents or compositions of the invention may be delivered once every 2-4 hours, or once every 4-6 hours, or once every 6-12 hours, or once every day over a course of treatment.

In some embodiments a course of treatment has a duration of about 1 day to about 1 week, or about 1-3 days, or about 3 days to about 1 week. In some embodiments a course of treatment may be repeated - i.e., there may be multiple cycles of treatment with breaks in between the cycles.

For those embodiments of the present invention where the active agent is camostat mesylate, in some of such embodiments an effective amount is about 0.05-1000 mcg/kg, or 0.5-100 mcg/kg, or about 5-10 mcg/kg, or about 0.05-0.5 mcg/kg, or about 0.5-1 mcg/kg, or about 1-10 mcg/kg, or about 10-100 mcg/kg, or about 100-1000 mcg/kg.

For those embodiments of the present invention where the active agent is nafamostat mesylate, in some of such embodiments an effective amount is about 0.005-1000 mcg/kg, or about 0.05-100 mcg/kg, or about 0.5-10 mcg/kg, or about 0.05-0.1 mcg/kg, or about 0.005-0.05 mcg/kg, or about 0.1-1 mcg/kg, or about1-10 mcg/kg, or about 10-100 mcg/kg, or about 100-1000 mcg/kg.

The invention may be further understood with reference to the following non-limiting Examples.

### EXAMPLES

### Example 1 - General Formulation Protocol

Exemplary, but non-limiting, protocols by which an aqueous composition comprising an active agent (e.g., camostat mesylate or nafamostat mesylate) can be formulated for inhalational delivery include the following.

Camostat mesylate or nafamostat mesylate is obtained at a suitable purity level (e.g., 99.8% purity - as assessed by ultrahigh pressure liquid chromatography (UPLC) combined with diode array and mass spectrometry detection (Acquity SQD system from Waters Inc., reverse phase C-18 Column, 1.7 mm, 2.1 X 100 mm column, using the gradient 5 to 95% acetonitrile in water, both containing 0.05% formic acid, 6 minutes run).

A stock solution of formulation agent (SBBCD or HPBCD or other agent) is first prepared in a sterile container.

For example, 100 mL of 50% SBBCD is prepared in a 100 mL volumetric flask by first placing 50 g of SBBCD powder in the measuring flask. Injection grade water (water for injection or “WFI”) is added in small portions with vigorous shaking to dissolve the powder. Additional WFI is then added to reach the 100 mL graduation before the measuring flask is closed with the appropriate glass stopcock and shaken upside down a few times to achieve homogeneity.

A precise amount, typically around 2.0 mg, of the active agent (e.g., camostat mesylate of nafamostat mesylate) is weighed using a precision balance (e.g., Delta Range, Mettler -Toledo), into a certified clean vial. The formulation is achieved by adding a minimal concentrated stock solution of formulation agent such as SBBCD solution prepared as above which is typically prepared at a 50% weight per volume (50% W:V) solution.

The resulting suspension of the active agent is then subjected to cycles of 1-minute sonication-vortex mixing until normal visual observation as well as observation under magnification indicates complete dissolution of the active agent. If a solution is obtained directly, then the experiment is repeated using smaller volumes with the aim or making a more concentrated solution. If a solution is saturated, then small amounts of formulation agent at final concentration are slowly added through precise pipetting of noted volumes, with continuous sonication-vortex mixing to reach dissolution. WFI is then added to bring final SBBCD concentration to 12.5%.

Concentrations (i.e., maximal dissolved concentrations) in milligrams per milliliter (mg/mL) are then deduced, and an additional control dissolution experiment is run to verify.

Using this method, a 19.0 mg/mL solution of camostat mesylate in WFI was obtained.

Following the same protocol as above, a 40% (w/v) solution of HPBCD in water for injection (WFI) was prepared, and camostat mesylate was then formulated in 10% (w/v) HPBCD in WFI to the level of 45.0 mg/ml.

Also following the same protocol as above, a 50% (w/v) solution of SBBCD in water for injection (WFI) was prepared using a volumetric flask and camostat mesylate was formulated in 12.5% (w/v) SBBCD in WFI to the level of 38.8 mg/ml.

### Example 2 - Particle Size Determination & Nebulization

Quality control assessments (such as by light scattering techniques) are performed post-formulation to assess the presence of any microparticles following microfiltration. Device calibration is conducted to ensure a sufficiently small droplet size from (e.g., from about 1 micron to about 10 microns) to ensure maximum delivery to lung. These parameters are also studied as a function of viscosity and drug concentration. Delivery of drugs to lungs via aerosolization is optimal with such droplet diameters. Sub-micron particles suspended in air may readily enter the lung alveolar space, but can remain suspended in the alveolar gas volume, and exit the alveolus during exhalation, and thus are not desirable for the intended use. On the other hand, larger particles can fall out of the air before reaching the lung alveolar epithelium.

Particle sizes of aerosolized formulated camostat mesylate and/or nafamostat mesylate were measured to assess suitability for pulmonary drug delivery using a Phase Doppler Analyzer (PDA) system (Dantec Dynamics A/S). Based on the optical system configuration, the accessible particle diameter measurement range is approximately 0.5 - 44 µm. Dantec Dynamics lists the stated uncertainty of this system to be approximately 2% of the range, which would translate to 0.87 µm.

Two methods of aerosolization were tested to determine whether camostat mesylate and nafamostat mesylate formulated in SBBCD and HPBCD would be compatible with aerosolization to yield particle sizes in a range that would be suitable for delivery to lungs. In one method, a jet nebulizer (Carefusion Airlife Jet Nebulizer) was loaded with formulated drug solutions with air flow entering the nebulizer at 5-15 L/min, and nebulized liquid particles exiting the flow system were measured by PDA and found to range from 1-10 microns in diameter. Particles generated by the jet nebulizer were smaller when air flow rates were higher. Thus, such a jet nebulizer delivery system could be employed for inhalational delivery of the formulations described herein to spontaneously breathing COVID-19 patients, with or without the attachment of the nebulizer to a rebreather reservoir bag.

In a second set of tests, formulated drugs were nebulized using an ultrasonic mesh nebulizer (Aerogen Solo). Such a system would allow nebulization of our formulations in-line with an airway circuit attached to a ventilator without the risk of environmental spillage of SARS-CoV-2 containing gases. Again, particle sizes were found to range from 1-10 microns in diameter.

Thus, both nebulizer systems have the potential to generate aerosolized particles from the camostat mesylate and nafamostat mesylate formulations described herein for delivery to subjects.

### Example 3 - Lyophilization

The stability of camostat mesylate and nafamostat mesylate formulations was studied at 2 temperatures - i.e. at 4° C. and ambient room temperature (~21° C.). A standard liquid chromatography mass spectrometry (LCMS) method was followed that uses ultraviolet and mass spectrometry in combination with Ultrahigh Pressure Liquid Chromatography (UPLC).

While even our non-lyophilized formulations were stable in the refrigerator at 4° C., the lyophilized versions had a longer shelf life.

Lyophilization was performed as follows: High concentration camostat mesylate and nafamostat mesylate formulations in SBBCD were flash-frozen using liquid nitrogen or dry ice-acetone and submitted to lyophilization using a bench top Virtis lyophilizer (operating at 40 mtorr vacuum and -105° C.) until a constant weight was obtained (typically 12 to 72 hours). The resulting white foam can be transferred to prelabeled clean vials, sealed and stored until needed.

Reconstitution of the lyophilized camostat mesylate or nafamostat mesylate formulations is achieved by adding the appropriate volume (e.g., the volume that was used in Example 1 to make the formulation) of WFI or other suitable solvent. Pulse vortexing and sonication cycles bring back the original liquid formulation.

### Example 4 - In Vivo Efficacy Studies

The in vivo efficacy of the compositions and inhalational treatment methods described herein is evaluated in a preclinical animal model such as mouse, rat, or guinea pig model, using the following in vivo luciferase reporter system. This system has the advantage of not requiring biosafety level 3 facilities - as are required for preclinical studies using live SARS-CoV-2 virus.

A luciferase protein, or a split luciferase protein pair, that requires the proteolytic activity of TMPRSS2 for luciferase activation is administered to the animal be inhalational delivery, and the small molecule luciferase substrate luciferin is also administered to the animal (the luciferin is given either systemically, or by inhalational delivery along with the luciferase). The luciferase or split luciferase has a TMPRSS2 substrate sequence from the SARS-CoV-2 spike protein, allowing the effects of the compositions of the present invention (administered to the animals by inhalational delivery) on TMPRSS2 activation of the viral spike protein to be assessed in vivo. In some variations of this method, the luciferase mutants are in a configuration that requires cleavage of protease substrate sequences for the luciferase to be activated. Other variations of this method utilize a luciferase intra-molecular complementarity assay in which the two halves are attached by the protease cleavage site linker, in such a conformation that cleavage is required to relieve the conformational strain such that the luciferase is activated. In another variation of this method, the luciferase is provided as two separate molecules/subunits, where one or both of the subunits has an attached inhibitor linked by the protease cleavage site, such that cleavage at the protease cleavage site relieves the luciferase domains to allow inter-molecular complementarity of the two fragments. These assays are performed using firefly luciferase, Renilla luciferase, Gaussia derived luciferase, luciferase from the deep shrimp Oplophorus (the latter is marketed as a split molecular complementation system branded NanoBit), or any suitable luciferase. In other variations of this method, a fluorescent protein is used - such as the jellyfish green fluorescent protein (GFP), or other fluorescent proteins tuned to other wavelengths (such as fluorescent proteins that can be imaged by a whole-body fluorescent imaging system, e.g., those fluorescent proteins that emit in the near infrared range of the light spectrum, to bypass background signal interference of hemoglobin and tissue autofluorescence).

A related method involves delivery of the luciferase (or fluorescent protein) TMPRSS2 protease biosensor encoded on an expression plasmid, and delivered inhalationally as naked DNA, or DNA formulated with cationic liposomes, or with polyethyleneimine, or poly-l-lysine (PLL), or as a lipid nanoparticle, or with cationic polysaccharides such as chitosan or other suitable formulations; or of luciferase delivered to be expressed by a non-pathological virus delivered inhalationally; or of luciferase delivered inhalationally as a version encoded by RNA formulated as a lipid nanoparticle, or other suitable formulations instead of direct inhalational delivery of luciferase (or fluorescent) protein.

Using these methods, the efficacy of the compositions and methods of the present invention is evaluated and confirmed in a clinically relevant animal model. Successful inhibition of the protease TMPRSS2 by a composition of the present invention quenches the luciferase in situ in these studies.

### Example 5 - Additional In Vivo Efficacy Studies

In a complementary strategy to that described in Example 4, preclinical studies are performed in which the luciferase (or fluorescent reporter protein) has a protease cleavable sequence (from SARS-CoV-2 spike protein) and activity of cellular protease TMPRSS2 inactivates the luciferase (or fluorescent reporter). These biosensor proteins (and substrates, in the case of a luciferase system) are delivered at the time of, or together with, or before the delivery of the compositions of the present invention. If the composition reaches the protease at a therapeutic concentration, then the protease cleavage site is blocked through protease inhibition, and the luciferase (or fluorescent reporter) signal is increased - as observed by whole-body luciferase imaging (or whole-body fluorescent imaging, in the fluorescent reporter version).

A related method involves delivery of the luciferase (or fluorescent protein) TMPRSS2 protease biosensor encoded on an expression plasmid, and delivered inhalationally as naked DNA, or DNA formulated with cationic liposomes, or with polyethyleneimine, or poly-1-lysine (PLL), or as a lipid nanoparticle, or with cationic polysaccharides such as chitosan or other suitable formulations; or of luciferase delivered to be expressed by a non-pathological virus delivered inhalationally; or of luciferase delivered inhalationally as a version encoded by RNA formulated as a lipid nanoparticle, or other suitable formulations instead of direct inhalational delivery of luciferase (or fluorescent) protein.

Using these methods, the efficacy of the compositions and methods of the present invention is evaluated and confirmed in second a clinically relevant animal model.

We claim:

1. A pharmaceutical composition suitable for administration to a human subject by inhalational delivery, the composition comprising:
a. camostat mesylate or nafamostat mesylate, and
b. a β cyclodextrin.

2. The pharmaceutical composition of claim 1, wherein the β cyclodextrin is sulfobutyl-β-cyclodextrin (SBBCD) or hydroxypropyl-β-cyclodextrin (HPBCD).

3. The pharmaceutical composition of claim 2, wherein the composition is an aqueous solution comprising camostat mesylate or nafamostat mesylate at a concentration of about 30-50 mg/ml in about 5-15% w/v HPBCD or SBBCD.

4. The pharmaceutical composition of claim 3, wherein the composition is an aqueous solution comprising camostat mesylate or nafamostat mesylate at a concentration of about 45 mg/ml in about 10% w/v HPBCD.

5. The pharmaceutical composition of claim 3, wherein the composition is an aqueous solution comprising camostat mesylate or nafamostat mesylate at a concentration of about 39 mg/ml in about 12.5% w/v SBBCD.

6. The pharmaceutical composition of any of the previous claims, further comprising one or more excipients suitable for inhalational delivery.

7. The pharmaceutical composition of claim 6, wherein the excipient is a co-solvent, a preservative, a chelating agent, a buffer, a pH regulator, a tonicity regulator, an amino acid, a carbohydrate, a synthetic polymer, a surfactant, or a preservative.

8. The pharmaceutical composition of any of the preceding claims, further comprising a propellant.

9. The pharmaceutical composition of any of the preceding claims in aerosol form.

10. The pharmaceutical composition of claim 9, wherein the aerosol form comprises liquid droplets of from about 1 to about 10 microns in diameter.

11. The pharmaceutical composition of any of claims 1-8 in lyophilized form.

12. The pharmaceutical composition of any of claims 1-11, wherein the composition is stable at 4° C.

13. The pharmaceutical composition of any of claims 1-11, wherein the composition is stable at 21° C.

14. A method of preparing a composition suitable for administration to a subject, the method comprising reconstituting a lyophilized composition according to claim 11 in a solvent to form a reconstituted composition suitable for inhalational delivery to a subj ect.

15. The method of claim 14, wherein the reconstituted composition further comprises an excipient selected from the group consisting of: a preservative, a chelating agent, a buffer, a pH regulator, a tonicity regulator, an amino acid, a carbohydrate, a synthetic polymer, a surfactant, or a preservative.

16. The method of claim 14 or claim 15, wherein the reconstituted composition comprises camostat mesylate or nafamostat mesylate at a concentration of about 30-50 mg/ml in about 5-15% w/v HPBCD or SBBCD.

17. The method of claim 14 or claim 15, wherein the reconstituted composition comprises camostat mesylate or nafamostat mesylate at a concentration of about 45 mg/ml in about 10% w/v HPBCD.

18. The method of claim 14 or claim 15, wherein the reconstituted composition comprises camostat mesylate or nafamostat mesylate at a concentration of about 39 mg/ml in about 12.5% w/v SBBCD.

19. A device for administration of a pharmaceutical composition to a subject by inhalational delivery, the device comprising a pharmaceutical composition according to any of claims 1-13.

20. The device of claim 19, wherein the device is a nebulizer, an inhaler, an atomizer or a vaporizer.

21. A method of treating COVID-19 in a subject, the method comprising administering to a subject in need thereof an effective amount of a pharmaceutical composition comprising camostat mesylate or nafamostat mesylate.

22. A method of treating COVID-19 in a subject, the method comprising administering to a subject in need thereof an effective amount of a pharmaceutical composition according to any of claims 1-13.

23. The method of claim 22, wherein the composition is administered to the subject using a nebulizer, an inhaler, an atomizer, or a vaporizer.

24. The method of claim 22, wherein the composition is administered to the subject using a ventilator or via intubation.

25. The method of any of claims 21-24, further comprising administering camostat mesylate or nafamostat mesylate to the subject by a systemic route.

26. The method of any of claims 21-24, further comprising administering camostat mesylate or nafamostat mesylate to the subject by oral administration.

27. The method of any of claims 21-24, further comprising administering camostat mesylate or nafamostat mesylate to the subject by intravenous administration.

28. The method of any of the claim 21-27, wherein the subject has tested positive for SARS-CoV-2.

29. The method of any of claims 21-28, wherein the subject is exhibiting one or more symptoms of COVID-19.

30. The method of any of claims 21-28, wherein the subject is not exhibiting symptoms of COVID-19.

31. The method of any of claims 21-28, wherein the subject is critically ill.

32. The method of any of claims 21-27, wherein the subject is intubated and/or on a ventilator.

33. The method of any of claims 21-28, wherein the subject is elderly, has hypertension, has lung disease, has cancer or is immunosuppressed or immunocompromised.
